SEQUENCE LISTING (8 sequences)

SEQ ID NO: 1 - protein, 17 aa, Artificial Sequence
  A peptide capable of inducing apoptosis.
AMAGPHPVIVITGPHEE

SEQ ID NO: 2 - protein, 13 aa, Artificial Sequence
  A peptide capable of inducing apoptosis.
KLAKLAKKAKLAK

SEQ ID NO: 3 - protein, 11 aa, Artificial Sequence
  Portion of a vector insert.
HGEFGGDDEVD

SEQ ID NO: 4 - protein, 12 aa, Artificial Sequence
  Portion of a vector insert.
HHHHHHGRGDNP

SEQ ID NO: 5 - protein, 23 aa, Artificial Sequence
  Portion of a vector insert.
HHHHHHGKLAKLAKLAKKAKLAK

SEQ ID NO: 6 - protein, 23 aa, Artificial Sequence
  Portion of a vector insert.
HHHHHHAMAGPHPVIVITGPHEE

SEQ ID NO: 7 - protein, 4 aa, Artificial Sequence
  A caspase-3 cleavage site.
DEVD

SEQ ID NO: 8 - protein, 9 aa, Artificial Sequence
  Portion of a vector insert.
HHHHHHGEF

(19) United States
(12) Patent Application Publication
(10) Pub. No.: US 2003/0035789 A1
(43) Pub. Date: Feb. 20, 2003

(54) Compositions and methods for caspase-induced apoptosis

(76) Inventor: Haval Shirwan, Louisville, KY (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/171,417

(22) Filed: Jun. 13, 2002

Related U.S. Application Data
(63) Continuation of application No. PCT/US00/34554, filed on Dec. 18, 2000.
(60) Provisional application No. 60/171,183, filed on Dec. 16, 1999.

(51) Int. Cl. A61K 38/48; A61K 38/20; A61K 38/19; C12P 21/02; C12N 5/06; C12N 9/64

(52) U.S. Cl. 424/85.1; 424/94.64; 424/85.2; 435/69.1; 435/69.5; 435/348; 435/226; 435/320.1

(57) ABSTRACT

This invention provides methods and compositions for caspase-induced apoptosis. Chimeric proteins are provided that target cells in which apoptosis is to be induced. The chimeric proteins of this invention activate endogenous caspase or provide caspase units that cause cell death. Methods are provided for the use of the chimeric proteins of this invention for in vivo and ex vivo therapies to treat pathological conditions.

### FIELD OF THE INVENTION

The present invention relates to the induction of caspase-induced apoptosis to eliminate activated lymphocytes for the relief of autoimmune diseases, allergies and to induce tolerance to allografts.

### BACKGROUND OF THE INVENTION

Apoptosis or programmed cell death is known to be involved in developmental processes that lead to growth and maturation in all animals from the tiny worm Caenorhabditis elegans up to and including humans. Normally, apoptosis is strictly regulated by physiological signals that lead to orderly development in various tissues. All cells have the capacity to undergo apoptosis, but in normal conditions, the apoptotic mechanisms are in the “off” mode. In pathological conditions, it would be advantageous to induce carefully targeted and controlled apoptosis in cells. Such conditions include those in which the immune system is dysfunctional, such as autoimmune diseases and allergies, or in which it is desirable to make the immune system less functional, as in allotransplantation. It is also desirable to induce apoptosis in undesirable cells, such as benign or malignant tumors.

The immune response is regulated by the interaction of several different cell types which react to the presence of foreign antigens. Adaptive immune response is critical to the survival of vertebrates in an environment full of pathogenic microorganisms. Individuals lacking an immune response through inborn genetic defects, exposure to chemotherapeutic agents or through infection by such viruses as human immunodeficiency virus (HIV), succumb to infections that an individual with a healthy immune response system would readily survive. However, the immune system is not always beneficial to the organism. Its dysregulation leads to autoimmunity and tumors. The immune system also serves as a barrier for the transplantation of beneficial foreign antigens such as those comprising an organ taken from another individual, a process that can replace failed organs in end-terminal diseases and treatment of a variety of hematopoietic disorders that can be treated with bone marrow transplants. For transplantation to be successful, it is necessary either to suppress the adaptive immunity or to “teach” the recipient's immune system to accept these foreign antigens as native.

T cells are the most critical cells of adaptive immunity as they orchestrate effector mechanisms of the adaptive immune system. The immune response to foreign antigens is initiated by naive T cells that use clonally-expressed T cell receptors (TCRs) to recognize the antigens such as peptides presented by self-major histocompatibility complex (MHC) molecules. This recognition reaction (signal 1), when accompanied by costimulatory signals (signal 2) provided by antigen-presenting cells (APCs), results in full T cell activation. Upon activation, T cells expand and differentiate into effector cells that evoke immunological mechanisms responsible for the clearance of antigens from the system. A period of death then follows during which most of the activated T cells undergo apoptosis-mediated “activation-induced cell death” (AICD) and effector activity subsides. Apoptosis is a complex process that involves a series of extra- and intracellular signals, that converge on the activation of enzymes called caspases, that commit the cell to apoptosis. Caspase-3 is the most critical “executioner” enzyme of the apoptosis pathway as it is the most downstream effector caspase that executes apoptosis by cleaving a series of cellular proteins needed for cell survival. Synthetic peptides containing selected motifs have recently been shown to activate caspase-3 in the cell without a requirement for signals transduced via the death receptors/ligand interactions.

Transplantation of vascularized grafts has become an important and effective therapeutic alternative for patients with selected terminal diseases. The transplantation of organs between generically different patients (“allografts”), is, however, limited by the ability to control the immunological recognition and rejection of the graft by the recipient. Pharmacological agents have been used more commonly as immunosuppressive regimens for prevention of allograft rejection. Although these drugs are effective in reducing the severity of rejection episodes, they are nonspecific and fail to create a state of permanent graft-specific tolerance. Continuous exposure of the recipient to these immunosuppressive agents to prolong graft survival is therefore, associated with a significantly increased risk of opportunistic infections and malignancies. Additionally, these nonspecific immunosuppressive agents can induce serious and undesirable pharmacological side effects in the host.

These undesired side effects often outweigh the benefits for patients with chronic, long-term autoimmune diseases, in which, for unknown causes, the body identifies certain parts of itself as “foreign” and launches an adaptive immune attack. Autoimmune diseases include lupus erythematosis, in which DNA of various tissues is the immune target; MS, in which the myeloid sheath of nerves is destroyed by immune reaction; and diabetes Type I, in which the pancreatic cells producing insulin are destroyed. Amyotrophic lateral sclerosis, thyroiditis and rheumatoid arthritis are also considered to be autoimmune in nature. It would be very desirable to be able to “teach” the immune system to tolerate the “foreign” self-antigen.

Some pathologies may not be due to a dysfunctional immune system per se, but the symptoms are due to a very active, uncontrolled immune system activity. Included in this group are allergies, asthma, and septic shock. The patient's distress would be alleviated by controlled elimination of activated immune cells.

In addition to pathological conditions caused by a dysfunctional immune system, there are other conditions in which a patient would benefit if certain cells are targeted for apoptosis. Chief among these are the various pathologies that can be lumped as tumors, either benign or malignant. It would be desirable to use an apoptotic method to eliminate these rogue cells.

The need remains to develop more selective and long-lasting methods to regulate the immune response and to eliminate undesired cells such as tumor cells.

### SUMMARY OF THE INVENTION

This invention provides compositions and methods to regulate apoptosis. The compositions include chimeric proteins capable of causing the induction of endogenous caspase or the administration of active caspase subunits. These compositions comprise (1) a vector comprising an affinity molecule targeted to the cell selected for apoptosis and (2) an insert that comprises a reactive portion of a caspase or a peptide that induces the activation of endogenous caspase in the targeted cell. When the target is an immune cell, the preferred affinity molecule is IL-2 and the preferred insert is the p12 or p17 subunit of Caspase-3. The methods of the invention teach those of skill in the art to construct chimeric proteins comprising other affinity molecules and other caspases. The methods of the invention also include in vivo and ex vivo induction of apoptosis of activated immune cells for the induction of tolerance to auto- and allo-antigens.

By target cell is meant any cell that has a unique surface protein that is a ligand which acts as receptor for a cell signal or an antibody or parts of an antibody, which protein is internalized when reacted with its ligand. Target cells includes leukocytes, activated leukocytes and tumor cells.

By caspase-induced apoptosis is meant cell death caused by internalization of a caspasse-related molecule which definition applies to caspase, active caspase subunit or caspase activating factor that activates endogenous caspase.

By production cell is meant any cell capable of producing caspase or active caspase subunits. E.coli, yeast and other microorganisms may be used as production cells. Surprisingly, it has been found that Drosophila cells can produce human caspase-related molecules.

One embodiment of the present invention is the induction of tolerance to allografts. It is known that the rejection of allografts is an immunological phenomenon initiated by the graft recipient's T cells, responding to the histocompatibility antigens on the graft. Upon stimulation, the clones of graft-specific T cells are activated, resulting in proliferation and the production of cytokines, which prolong and intensify the reaction. Experimental animals lacking T cells, either due to congenital immunodeficiency or experimental manipulations, can be conditioned to accept allografts expressing strong histocompatibility differences for indefinite periods of time. T-cell recognition of alloantigens via clonally-expressed T-cell recognition transduces an antigen-specific signal (signal 1) that initiates a series of intracellular biochemical events culminating in T-cell activation. Signal 1 alone, however, is not sufficient for full T cell activation; additional co-stimulatory signals (signal 2) provided by antigen-presenting cells (APC's) are also required for generation of the effector mechanisms of allograft rejection. The transduction of signal in the absence of signal 2 may result in functional silencing (anergy) or physical elimination (apoptosis) of naive T cells. Upon activation, T cells undergo a state of antigen-driven proliferation that allows up to a 1200-fold clonal expansion. A period of death then ensues during which more than 95% of the activated T cells undergo apoptosis (programmed cell death) while the remaining cells differentiate into memory cells as the amount of antigen in the system declines.

Apoptosis or “programmed cell death” plays a central role in both the development and homeostasis of multicellular organisms. Apoptosis can be induced by multiple independent signaling pathways that converge upon a final effector mechanism consisting of multiple interactions between several “death receptors” and their ligands, which belong to the tumor necrosis factor (TNF) receptor/ligand superfamily. The best characterized death receptors are CD95 (“Fas”), TNFR1 (p55), death receptor 3 (DR3 or Apo3/TRAMO), DR4 and DR5 (apo2-TRAIL-R2). A final effector mechanism is mediated by the caspase group of proteins. In addition, it has been found that certain natural or synthetic peptides also induce apoptosis when delivered into a cell, possibly by activation of endogenous caspases. This latter group includes RGD, AMAGPHPVIVITGPHEE, KLAKLAKKAKLAK, cytochrome c (“cyto”) and DIABLO/SMAC (“SMAC”), collectively referred to as caspase activating factors (CAF).

Caspases play the most critical role in apoptosis and have been found in organisms ranging from Caenorhabditis elegans to humans. Over 13 mammalian caspases have been identified to date. Caspases share similarities in amino acid sequence, structure, and substrate specificity. They are all expressed as 30 to 50 kDa inactive proenzymes that contain three domains; and N-terminal prodomain followed by a large (20 kDa) and a small (˜10 kDa) subunit. Physical separation of these three domains by proteolytic cleavage is followed by association of the large and small subunits to form a heterodimer. Two heterodimers may then associate to form a tetramer consisting of two catalytic sites that appear to function independently. Multiple apoptotic signals and cofactors are involved in the initial activation of the initiator caspases that are critical to proteolytic cleavage and activation of the downstream caspases and the execution of the apoptotic machinery. Each step in this sequence, up to the point at which caspase-3 is activated, is susceptible to regulation.

Caspase-3 was selected as a preferred agent for inducing apoptosis in graft-specific, activated T cells. Caspase-3 is a key enzyme, both necessary and sufficient, in the apoptotic pathway as its activation leads directly to cell death. For example, cross-linking of the caspase-3 with synthetic peptides results in its activation and apoptosis of the cell, showing that active caspase-3 on its own is sufficient for the apoptotic process and can bypass intracellular checkpoints that, under normal physiological circumstances, limit apoptosis. Caspase-3 proenzyme contains an N-terminal prodomain followed by a p17 domain that contains the catalytic cysteine residue, and a p12 domain. There are two caspase cleavage sites situated between the prodomain/p17 and the p17/p12 domains. The proenzyme form is inactive. During apoptotic signaling, however, it is cleaved by upstream caspases, such as caspase-8 in T cells, resulting in the loss of inactive prodomain and formation of and active p17/p12 heterodimer and/or heterotetramer. Caspase-3 can autocatalyze the cleavage of inactive procaspase-3, thereby further augmenting the apoptotic signal. It has been found that CAF can activate procaspase without the addition of active caspase-3.

If it is desirable to allow additional regulation before the final commitment to death (activation of caspase-3) any of the other caspases may be linked to a delivery vehicle, according to the techniques and methods disclosed in this application.

A delivery vehicle for the purposes of this invention is defined as a ligand to any receptor uniquely present, or present at uniquely high levels, on the cell to be trageted for apoptosis. When the target is activated immune cells such as T cells, this class of compounds includes the death receptor/ligand pairs as set forth the in background of the invention and the interleukins. Interleukins (IL) belong to the class of compounds generally termed cytokines. Receptors to the ILs are induced when immune cells are activated. Among the delivery vehicles especially useful in this invention when immune cells are the target for apoptosis are IL-2, IL-4, IL-6, IL-8, IL-10, IL-12, IL-15, TNF-α and TGF-β.

More generally, delivery vehicles may be polyclonal or monoclonal antibodies against cell surface proteins. More advantageously, the binding domains, V_{light} and V_{heavy}, of anitbodies may be constructed by methods well known in the art, and ligated to caspase subunits or CAF's. A cell surface protein commonly found on cancer cells can be used in a construct when cancer cells are targeted.

Interleukin 2 (IL-2) plays a central role in clonal expansion of activated T lymphocytes and is a preferred delivery vehicle. T cell recognition of foreign antibodies by the TCR (signal 1) accompanied by costimulatory signals (signal 2) results in the induction of IL-2 and high affinity IL-2 receptor (IL-2R) in the T cell. Signals transduced by the interaction of IL-2 with high affinity IL-2R in an autocrine or paracrine fashion results in T cell activation, proliferation, and differentiation into effector function. Activated T cells, in turn, synthesize and secrete a series of cytokines that work on other cells of the immune system for intensification and generation of an effective immune response to foreign antigens.

IL-2 mediates multiple biological processes, including growth and differentiation of B cells, generation of lymphokine-generated killer cells, augmentation of native killer (NK) cells, and proliferation and maturation of oligodendroglial cells. Furthermore, IL-2 can also regulate the cell growth, program mature T cells for apoptosis and rescue T cells from anergy. Consistent with these far-ranging functions, IL-2 delivers various signals to a wide range of cell types via interaction with its high affinity receptor. The binding of IL-2 to its high-affinity IL-2R is followed by the internalization of the complex via two pathways; one dependent of clathrin-coated pits and the other independent of clathrin-coated pits. After endocytosis of the complex, the α-chain of IL-2R recycles back to the plasma membrane while IL-2, and the β- and γ-chains are routed towards late endocytotic compartments via discrete internalization and degradation motifs.

The selective expression of high-affinity IL-2R in activated T lymphocytes has led to a series of approaches using IL-2R-directed therapy for prevention of autoimmunity, graft rejection, and certain neoplastic diseases. A number of toxins have been conjugated to delivery vehicles such as IL-2 or to antibodies to the α-chain of IL-2R which specifically target activated T cells. When the complex is internalized, the toxin is freed to cause cell death. For example, IL-2-PE40, (P. exotoxin-IL-2 chimeric protein) has been shown to be highly toxic for leukemic cell lines as well as for activated normal T cells. The IL-2-toxin fusion proteins were found to be potent immunosuppressive agents capable of preventing alloreactivity, including allograft rejection, in several different experimental settings. However, these agents are not free of side effects.

Apoptosis of activated T cells results in tolerance to allografts and xenografts, including bone marrow and other organ transplantation. Purging of activated T cells also relieves the symptoms of allergies and other immune-induced diseases. Included in the latter are auto-immune disorders such as multiple sclerosis, lupus erythematosus, type I diabetes, sarcoidosis and rheumatoid arthritis. Many disorders, including some tumors, are dependent on lymphocyte functions that lead to persistence of the disorder. Many hematological disorders could be treated with bone marrow stem cell transplants if the immune response could be regulated so as to induce tolerance to the foreign stem cells. Among these disorders are leukemias, lymphomas, aplastic anemia, sickle cell and Cooley's anemia and the like. All of these disorders may be controlled permanently or temporarily by apoptosis of activated immune cells, including T cells.

This invention discloses strategies for immunoregulation which comprise the construction of chimeric cDNAs encoding the functional portions of a delivery vehicle that specifically targets activated T cells, operably linked to an apoptosis-inducing molecule. In addition to the chimeric protein with caspase-derived inserts, it has surprisingly been found that two additional classes of intracellular proteins are useful in promoting apoptosis. These classes are exemplified by (a) cytochrome c (“cyto”), which is found in mitochondria, and (b) DIABLO/SMAC (“SMAC”), a newly discovered protein that binds to the endogenous inhibitor of apoptosis to clock its activity, thereby promoting apoptosis.

Table I is a summary of constructs. Choice of constructs may be based on factors such as : the nature of the foreign antigen provoking adaptive immunity; whether relief sought will be temporary or permanent; whether a commitment to death is desired or addition downstream regulation of apoptosis is preferred. It is to be understood that the constructs listed are representative of the invention only and are not limitations. Those skilled in the art can readily, without undue experimentation, make constructs with any of the 14 caspases, CAFs, cyto c, or SMAC operably linked to a delivery vehicle.

TABLE I

| Vector | Insert | Made | Function | Application |
|---|---|---|---|---|
| pIL2 | IL-2-6HisGEF | + | Cell Growth | Maintain IL-2-dependent cells, |
| | | | | use as a control in our |
| | | | | experimental systems |
| pIL2-p17 | IL-2-His-GEFGGDDEVD-p17 | + | Apoptosis | Tolerance to allografts, |
| | | | | xenografts, bone marrow |
| | | | | transplantation, treatment of |
| | | | | autoimmune diseases, |
| | | | | allergies, other immune- |
| | | | | induced diseases, and |
| | | | | lymphocyte-dependent |
| | | | | disorders, including tumors |
| pIL2-p12 | IL-2-His-GEFGGDDEVD-p12 | + | Apoptosis | Tolerance to allografts, |
| | | | | xenografts, bone marrow |
| | | | | transplantation, treatment of |
| | | | | autoimmune diseases, |
| | | | | allergies, other immune- |
| | | | | induced diseases, and |
| | | | | lymphocyte-dependent |
| | | | | disorders, including tumors |
| p17 | R-p17-6His | + | Apoptosis | Tolerance to allografts, |
| | | | | xenografts, bone marrow |
| | | | | transplantation, treatment of |
| | | | | autoimmune diseases, |
| | | | | allergies, other immune- |
| | | | | induced diseases, and |
| | | | | lymphocyte-dependent |
| | | | | disorders, including tumors |
| p12 | RS-6His-p12 | + | Apoptosis | Tolerance to allografts, |
| | | | | xenografts, bone marrow |
| | | | | transplantation, treatment of |
| | | | | autoimmune diseases, |
| | | | | allergies, other immune- |
| | | | | induced diseases, and |
| | | | | lymphocyte-dependent |
| | | | | disorders, including tumors |
| pIL2-RGD | IL-2-6His-GRGDNP | + | Apoptosis | Tolerance to allografts, |
| | | | | xenografts, bone marrow |
| | | | | transplantation, treatment of |
| | | | | autoimmune diseases, |
| | | | | allergies, other immune- |
| | | | | induced diseases, and |
| | | | | lymphocyte-dependent |
| | | | | disorders, including tumors |
| pIL2-KLA | IL-2-6His-GKLA | − | Apoptosis | Tolerance to allografts, |
| | KLAKLAKKAKLAK | | | xenografts, bone marrow |
| | | | | transplantation, treatment of |
| | | | | autoimmune diseases, |
| | | | | allergies, other immune- |
| | | | | induced diseases, and |
| | | | | lymphocyte-dependent |
| | | | | disorders, including tumors |
| pIL2-VIV | IL-2-6His- | − | Immuno- | Tolerance to allografts, |
| | AMAGPHPVIVITGPHEE | | suppression | xenografts, bone marrow |
| | | | | transplantation, and treatment |
| | | | | of autoimmune diseases |
| pIL-2-cyto | IL-2-6His-cyto | − | Apoptosis | Tolerance to allografts, |
| | | | | xenografts, bone marrow |
| | | | | transplantation, treatment of |
| | | | | autoimmune diseases, |
| | | | | allergies, other immune- |
| | | | | induced diseases, and |
| | | | | lymphocyte-dependent |
| | | | | disorders, including tumors |
| pIL-2- | IL-2-6His-SMAC | − | Apoptosis | Tolerance to allografts, |
| SMAC | | | | xenografts, bone marrow |
| | | | | transplantation, treatment of |
| | | | | autoimmune diseases, |
| | | | | allergies, other immune- |
| | | | | induced diseases, and |
| | | | | lymphocyte-dependent |
| | | | | disorders, including tumors |

The dosage necessary to induce apoptosis is dependent on the affinity and specificity of the delivery vehicle and the activity of the apoptotic agent. Ten to 100 molecules of the preferred embodiment per cell, IL-2/caspase-3, is sufficient for the apoptotic effect, while as much as 1,000 to 10,000 molecules of less active constructs may be necessary. The choice of delivery vehicle and apoptotic agent will depend on the activity desired. The construction of the preferred embodiment IL-2/caspase-3 and IL-2CAF is schematically described in FIG. 1 and its mode of action is shown in FIG. 2.

A preferred production cell for the production of chimeric proteins encoded by the DNA constructs is the Drosophila system that is commercially available. However, those skilled in the art of producing chimeric proteins will recognize that many other expression systems and vectors are suitable for production of the chimeric proteins of this invention. Included in these other systems are Escherichia coli, yeast and mammalian cell cultures.

Those of skill in the art will readily recognize that, using the methods disclosed in this invention, cells other than leukocytes can be caused to undergo apoptosis. Cell surface proteins specific to various cells are well known in the art. For example, cancer cells have tumor antigens against which specific antibodies may be produced. The DNA coding for these antibodies can be ligated to a caspase-derived DNA sequence, such as p12 or p17, or to the DNA coding for an activator of endogenous caspase such as cytochrome c. More advantageous, the specific binding domains of the antibody (the variable regions of the two protein chains, V_{light} and V_{heavy}) can be utilized. Methods of obtaining these domains have been well-known in the art. See, for example, Bird et al., Science 242, 423-426 (1988) or Huston, J. S. et al., Proc.Nat.Acad.Sci.USA 85, 5879-5883 (1988).

### DESCRIPTION OF THE DRAWINGS

FIG. 1 shows construction of Il -2/caspase-3 and IL-2-2C/CAF.

FIG. 2 shows the mode of action of the construct of FIG. 1.

FIG. 3 shows the PCR products.

FIGS. 4 and 5 show the EcoR1 digestion of positive clones.

FIG. 6 shows the production of chimeric protein.

FIG. 7 shows a schematic representation of chimeric proteins.

FIG. 8 shows growth of IL-2- dependent CTLL-2 cells in the presence of chimeric proteins.

FIG. 9 shows the activation of endogenous Caspase-3.

FIG. 10 shows induced apoptosis in activated lymphocytes.

FIG. 11 shows the inhibition of alloreactive lymphocyte response by chimeric proteins.

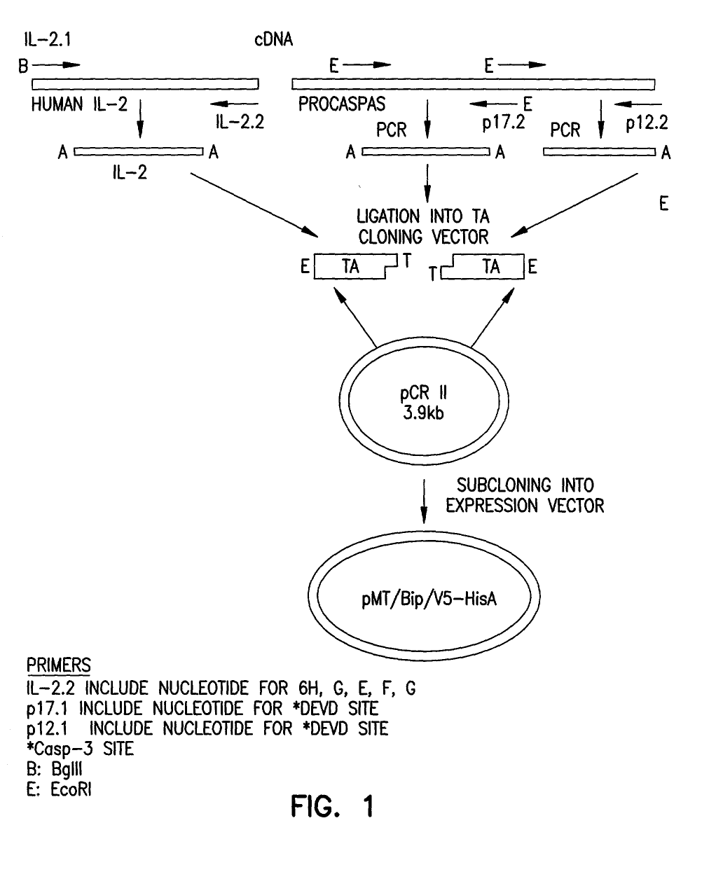

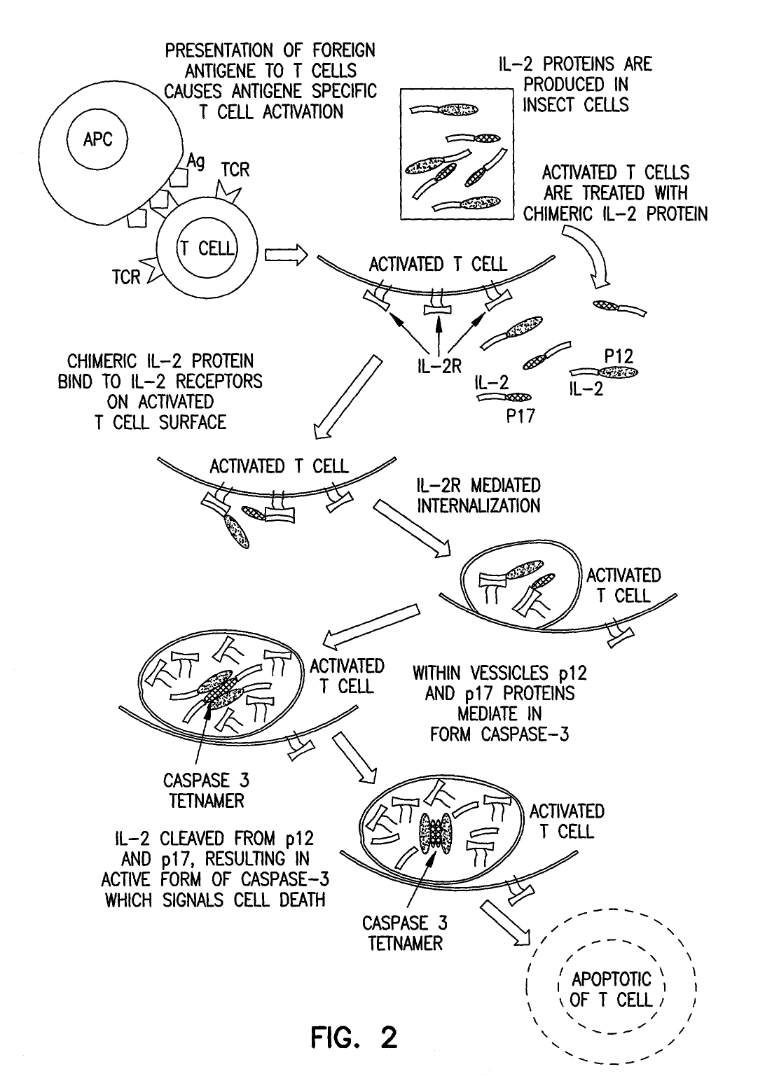

### DETAILED DESCRIPTION OF THE INVENTION

The experimental procedures described herein are representative of compositions and methods to regulate the immune system by manipulation of the caspase system. Following the teachings of this invention and using compositions known in the art, any of the 13 known caspases may be made into a construct with the delivery vehicle targeted to the cell of choice. The choice of caspase depends on whether a commitment to death with Caspase-3 is desired or whether a caspase higher in the cascade, that is, permitting subsequent regulation, is preferable. The delivery vehicle targeted can be any known ligand of any cell surface protein, such as a cytokine or antibody or fraction of antibody. Alternatively, a chimeric protein can be constructed that activates endogenous caspase. The DNA coding for these chimeric proteins can be used for gene therapy.

In general the access of the chimeric proteins to the cytosol of the target cell may be facilitated by the simultaneous application of viral peptides, synthetic peptides and fusogenic pharmaceutical agents. These formulations are well known to those in the art.

In one embodiment, the targeted cell is activated lymphocytes and the result sought is allograft tolerance which can be obtained by application of the preferred embodiment, IL-2R-targeted caspase-3, via the delivery of an active form of caspase-3 using IL-2/p17, IL-2/p12 or IL-2R/CAF chimeric proteins into alloreactive immune cells, thereby causing death by apoptosis. These proteins will be injected into graft recipients in various combinations in order to induce donor-specific long-term survival and/or tolerance. Alternatively, the DNA encoding such chimeric proteins may be cloned into an expression vector coupled to a delivery vehicle such as IL-2 at the protein level and applied ex vivo or in vivo, leading to permanent transformation of activation recipient T cells in which apoptosis is induced.

In a second embodiment, the chimeric proteins causing apoptosis of activated immune cells will be used to construct an affinity column for ex vivo purging of immune cells to relieve the symptoms of disease caused by immune activation. These diseases include autoimmune diseases, allergies, asthma and toxic shock.

In a third embodiment, the methods of this invention will be applied to construct chimeric proteins comprising a caspase or CAF and a ligand of a surface protein unique to a cell which is to be subjected to apoptosis. The preferred ligand is an antibody directed against such unique cell surface protein or a binding domain of such antibody. These diseases include benign and malignant tumors.

1. Construction of Chimeric Genes for Expression in Production Cells

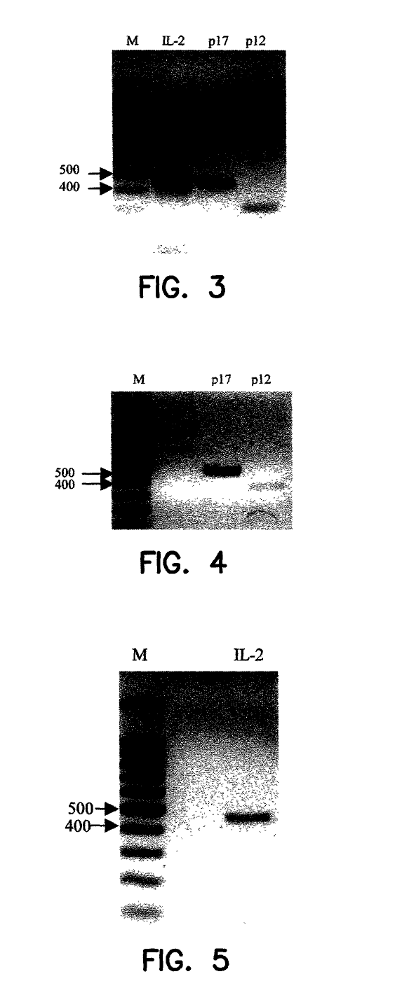

Total RNA was prepared from human Jurkat cell line and 2 μg of this RNA was converted into the first strand of DNA using oligo (dT)₁₈ as a primer for reverse transcriptase. One-tenth of this cDNA preparation was then amplified, using three sets of sense and antisense primers specific for human IL-2, p17 and p12 in three separate PCR amplifications. The IL-2 3′ antisense designed to include nucleotides encoding a caspase-3 cleavage site (DEVD) and an EcoR 1 site were in frame with IL-2 for construction. As shown in FIG. 3, these primers amplified DNA bands of expected sizes for all three genes of interest. These PCR products were then cloned into the TA cloning vector (Invitrogen, San Diego, Calif.) and a library prepared from this material was screened using the same oligonucleotide primers in PCR amplifications. The positive clones were digested with EcoR1 which has two sites surrounding the multiple cloning sites of the TA vector. As shown in FIGS. 4 and 5, EcoR1 digestion resulted in the release of expected size of fragment for each gene.

Functional cDNAs for the 6×His-IL-2 and IL-2/p17 molecules were subcloned in frame with the Drosophila BiP secretion signal in the pMT/Bip/NV5-HisA vector for expression in a high-yield insect expression system (DES™, Invitrogen). Recombinant vectors were transiently transfected into Drosophila S2 cells using calcium phosphate. Cells were then pulsed with copper sulfate to activate the inducible metallothionein promoter driving the expression of chimeric proteins. Culture medium was collected at various times post-activation, dialyzed to remove the copper sulfate, and analyzed for chimeric proteins using ELISA, Western blot and CTLL-2 proliferation assays.

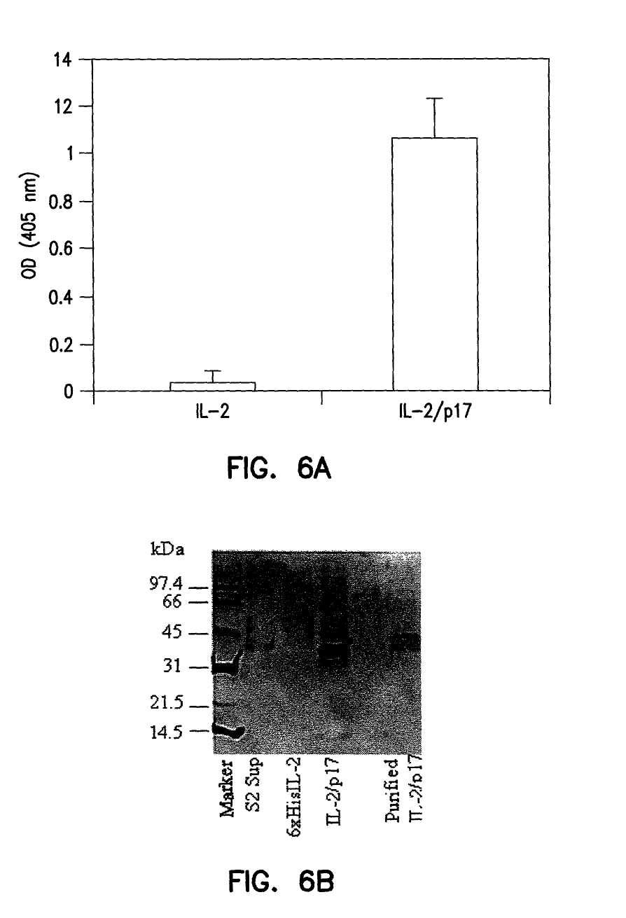

ELISA was performed by incubating culture supernatants in Ni-NTA-coated, 96-titer plates for absorption at 4° C. overnight, Ni-NTA specifically binds to 6×His-tagged proteins and as such confers specificity and sensitivity. After several washes with phosphate buffered salt solution (PBS), wells were incubated with various dilutions of rabbit anti-human procaspase-3 polyclonal antibody for one hour at room temperature. (PharMingen, San Diego, Calif.). Alkaline phosphatase-conjugated goat anti-rabbit IgG antibody was used for detection using a standard ELISA protocol. As shown in FIG. 6, supernatants from S2 transfectants expressed significant levels of chimeric IL-2/p17 protein. Culture supernatant from S2 sells transfected with the 6×His-IL-2 construct served as a negative control.

Supernatants were next analyzed in Western blots to demonstrate the presence of protein and to obtain information on its molecular weight. Antiserum against human procaspase-3 detected a molecule at ˜34 kDa in the supernatant of IL-2/p17 transfectants, but not in supernatants of 6×His-IL-2 transfected nor in untransfected S2 cells. This is the expected molecular weight for the chimeric IL-2/p17, as each protein contributes about 17 kDA to the total size.

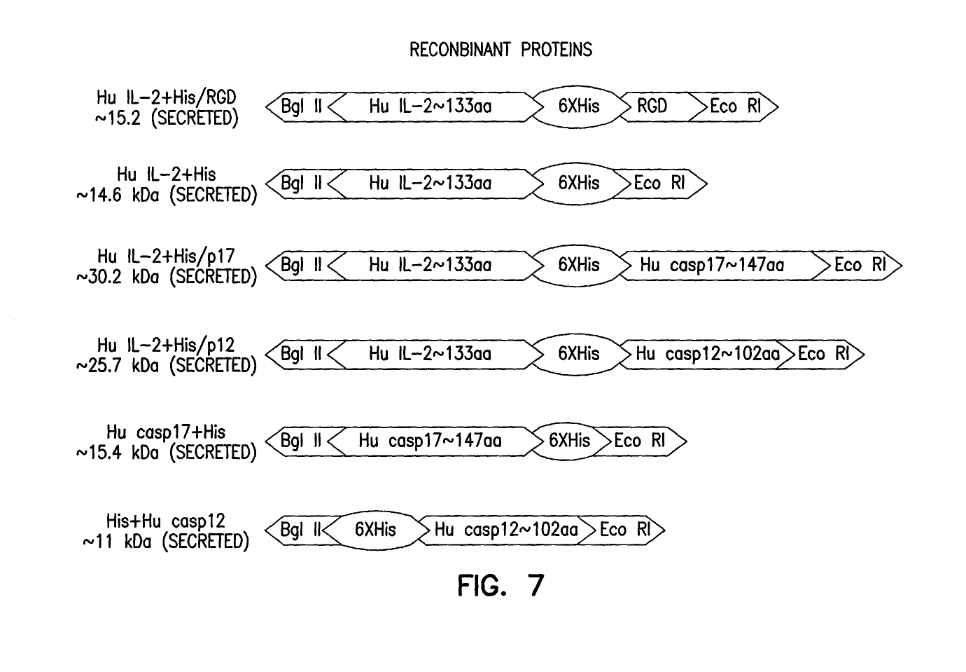

The other clones of Table I were constructed and characterized using similar methods. All indicated clones were sequenced and found to have the expected characteristics. FIG. 6 shows that the chimeric proteins retain the immunological characteristics of human caspase-3. Similar experiments show that the IL-2 moiety reacts with human anti-IL-2. FIG. 7 is a schematic representation of these chimeric proteins.

2. Chimeric Proteins Support the Growth of IL-2 Dependent CTLL-2 Cells

To demonstrate whether the IL-2 in chimeric proteins is functional, supernatants or purified proteins were tested for supporting the growth of IL-2-dependent CTLL-2 cell line, a mouse lymphoma line constitutively expressing IL-2R and dependent on IL-2 for growth.

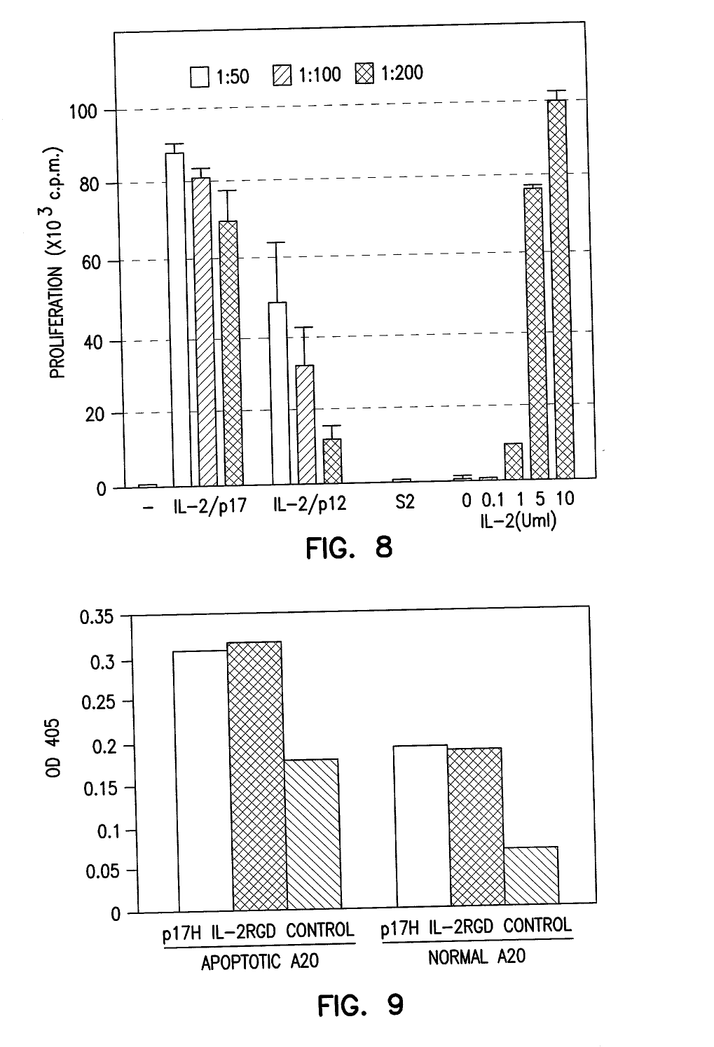

Recombinant proteins were tested on the CTLL-2 mouse lymphoma cell line that constitutively expresses IL-2R and is dependent on IL-2 for growth. Cells were cultured at 5000 cells/well in 200 μl total volume of medium in U-bottom 86-microtiter plates in the presence of various amounts of recombinant proteins individually or in various combinations, starting with concentrations equivalent to 10 U of IL-2 required for optimum cell growth in culture. Cultures receiving IL-2 and without IL-2 served as positive and negative controls, respectively. After 18 hour incubation, cells were pulsed with 1 μCi³H-thymidine/well for an additional eight hours. The proliferative response was determined after harvesting the cultures on glass-fiber filters and counting filter-associated radioactivity in a microplate scintillation counter. As shown in FIG. 8, IL-2/p12 and IL-2/p17 recombinant proteins supported the growth of CTLL-2 cells in a dose-dependent manner. Il-2/p17 appeared to be somewhat more active than IL-2/12 for supporting cell growth, although the supernatants were not normalized to a constant recombinant protein content. Transfectants expressed significant amounts of the recombinant proteins, corresponding to 250-2800 units of commercial IL-2 per ml of tissue culture medium. This attests to the efficiency of the DES™ system. These data provide direct evidence that (1) fusion with caspase-3 subunits does not interfere with the function of IL-2, (2) chimeric proteins individually are not toxic to the cell, and (3) chimeric proteins can be efficiently and functionally expressed using the DES™ system.

Recombinant proteins were tested on the CTLL-2 cell line. Cell lysates were prepared from normal mouse A20 or A20 cells treated with an apoptotic form of FasL as an external signal to induce apoptosis, Lysates were then incubated with the indicated chimeric proteins (FIG. 9) in the presence of Caspase-3 chromogenic substrate Z-DEVD-AFC for four hours. The cleavage of the substrate was then measured at 405 nm. Cell lysate alone served as the negative control. As shown in FIG. 9, recombinant proteins activated the endogenous Caspase-3 in lysates from unmanipulated cells and further augmented the activity of Caspase-3 in lysates from apoptotic cells, demonstrating that recombinant proteins comprising CAF are filly capable of activating endogenous Caspase-3.

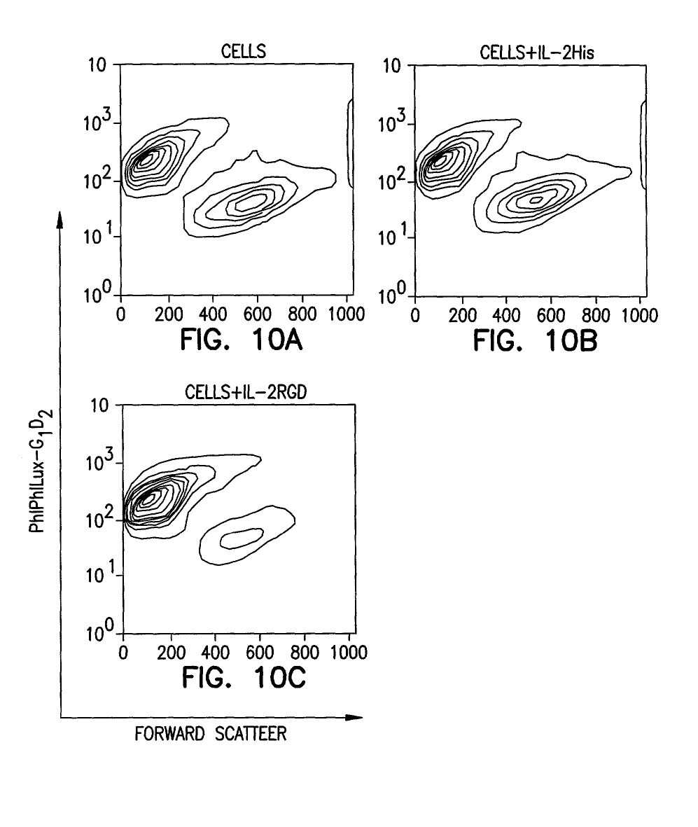

Next, the endogenous Caspase-3 was activated by specific delivery of caspase-inducing CAFs using cytokines, in this case IL-2, as a delivery vehicle into activated lymphocytes expressing IL-2 receptor. Rat splenocytes were activated using Concanavalin A (ConA) as a T-cell mitogen, for 18 hours. These cells were then washed extensively to remove the extra ConA and incubated with recombinant proteins at various doses and combinations for 18 hours. Cells were washed again and incubated with the Caspase-3 PhiPhiLux-GID2 (GDCDGI) fluorogenic substrate (10 μM; Ocolmmun, Inc., Gaithersburg, Md.) for one additional hour. Cells were then washed extensively and analyzed in FL1 channel for apoptotic activity. As shown in FIG. 10, recombinant IL-RGD caused significant apoptosis of activated lymphocytes as compared with IL-2His, the negative control (R1=43% vs. 72%). These data clearly demonstrate that the recombinant proteins can be internalized via IL-2R and activate endogenous Caspase-3 that commit the cell to death.

3. In vitro Test for Induction of Allotolerance

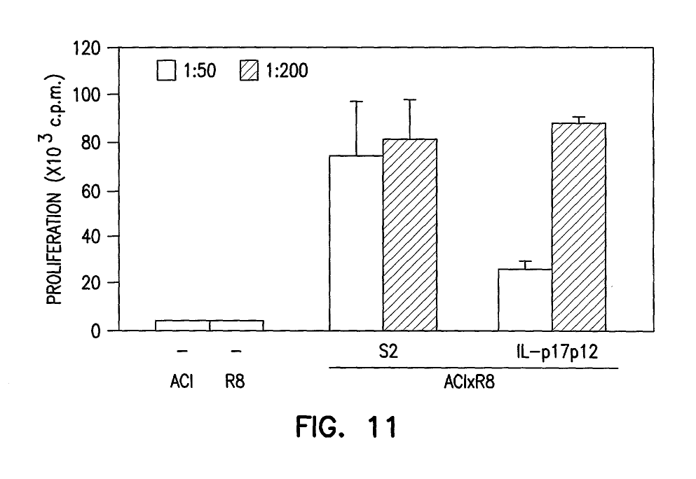

The efficacy of the chimeric proteins for induction of apoptosis in vivo will be tested for the induction of tolerance to cardiac allografts in three different rat strain combinations. In preparation for in vivo testing, the ability of chimeric IL-2/p12 and /p17 to inhibit alloreactive responses was tested in vitro in allogeneic mixed lymphocyte cultures as a model system. Splenocytes were harvested from several different rat strains with minor and MHC antigenic differences and were used in a standard MLR assay in the presence of various doses and combinations of recombinant proteins. Maximum inhibition was observed for MLR cultures incubated with a mixture of IL-2/p17/IL-2/p12 molecules, as shown in FIG. 11. S2 supernatant and cells alone (ACI and IU) served as controls. The data is the representative of three independent experiments.

The inhibitory effect of this regimen ranged within 60%-80% and was observed at 1:50 dilution of the supernatant tested. Chimeric molecules moderately inhibited the MLR response when used separately as compared with the control S2 supernatant. Further studies, however, are needed to optimize the dose and time of application for the most inhibitory effect. These data provide support for the hypothesis that the IL-2R-targeted delivery of caspase-3-activating protein molecules may serve as an effective means to specifically eliminate activated lymphocytes expressing high-affinity IL-2R.

4. In vivo Studies on the Inhibition of Alloreactivity.

Further studies are planned on the ACI-to-LEW mouse combination, which is disparate for minor and major histocompatibility antigens and a high responder combination that requires stringent regimens for tolerance induction and as such resembles human clinical situations. Furthermore, cellular immune responses, particularly those mediated by the direct pathway, play a major role in graft rejection in this model. The PVG.R8-to-PVG.IU model, on the other hand, involves disparity at one isolated class I, RT1.A^{a}, antigen that is sufficient to initiate vigorous immune responses that result in acute rejection in seven days. Humoral immune response plays a critical role in acute graft rejection in this model. The PVG.IU-to-PVG.R8 combination is also disparate for one isolated class I, RT1 .A^{u}, molecule that causes chronic rejection of cardiac allografts in this model. The indirect recognition pathway is an important component of alloreactive immune responses that mediate rejection in the PVG.R8 and PVG.1U models. The use of these three allograft models will test the efficacy of the chimeric proteins for induction of tolerance in T cells, primed by direct as well as indirect pathways, and in B cells. It is critical especially to test if these chimeric proteins prevent chronic rejection that is the main cause of late graft failures.

Intra-abdominal and cervical heterotopic cardiac grafts will be evaluated daily for function by palpation. Presensitization of prospective allograft recipients will be performed with i.p. injection of 1×10⁷ irradiated donor splenocytes or cervical grafts two weeks before intra-abdominal heterotopic transplantation. Graft recipients will be treated either i.p. or i.v. with 0.02 to 2.0, most preferably 0.25 μg, recombinant chimeric proteins at various times pre- and post-transplantation.

Graft survival will be assessed by palpation of the transplanted heart for acute rejection. Chronic rejection will be assessed by histological examination.

Based on the in vitro studies, it is expected that allotolerance will be induced in the animals treated with the chimeric proteins of this invention.

5. Ex vivo Apoptosis of Activated T Cells

In some clinical situations, it may be advisable to purge the patient of activated T cells without inducing a permanent effect. Many techniques are known for continual removal blood continuously from the body, subjecting it to treatment outside the body with subsequent return. A patient suffering from an autoimmune disease will especially benefit from leukophoresis, in which the white cells are continuously collected, followed by treatment with one of the chimeric proteins of this invention. Those activated T cells killed via apoptosis will be removed and the leukocytes returned to the patient. In this manner, the activating antigen contained in the lymphocytes and the activated lymphocytes are removed from the body of the patient, rather than being released into the bloodstream.

The isolated human lymphocytes can be caused to induce apoptosis by applying the methods of Example 3.

6. Induced apoptosis in other tissues.

In order to use the methods of this invention to induce apoptosis in tissues other than activated lymphocytes, it is necessary only to identify a cell surface protein that is unique to the target identified and that is internalized into the cell. Many such cell surface proteins are known. Antibodies against these proteins can be raised by techniques well known to those skilled in the art. Caspase subunits or compounds which activate endogenous caspase, such as cytochrome c, can be ligated to the antibody or the variable regions of the antibody and administered to induce apoptosis in the undesired cells.

7. Gene Therapy using the Chimeric Genes of this Invention.

The DNA constructs of examples 1 or 6 can be linked to delivery systems as a means of gene therapy. The DNA will be targeted for delivery into cells of interest to induce apoptosis, giving permanent regulation of the immune response.

This invention has been described in various preferred embodiments. Those skilled in the art will readily recognize that modifications, deviations or substitutions of the compounds or methods here disclosed may be made without departing from the spirit and scope of this invention. All such modifications, deviations and substitutions are considered to be within the scope of the claims of this invention. Any references cited within are hereby incorporated by reference.

What is claimed is:

1. A method for effecting caspase-induced apoptosis in a target cell comprising:
(a) transfecting a production cell with a vector comprising DNA encoding a protein that selectively binds to the target cell, operably linked to DNA encoding an caspase-related molecule that induces apoptosis when taken into the target cell, wherein the production cell is capable of transcribing and translating the DNA to produce chimeric proteins;
(b) recovering such chimeric proteins; and
(c) introducing an effective amount of the chimeric protein into the target cell.

2. The method of claim 1, wherein the cell is an activated lymphocyte and the caspase-related protein is Il-2, IL-4, IL-8, IL-10, IL-12, IL-15, TNF-α, GM-CSF or TGF-β and the apoptosis inducing molecule is one of caspase 1 to 14, or a caspase activating factor.

3. The method of claim 1, wherein the production cell is a Drosophila cell.

4. A chimeric protein comprising a protein that selectively binds to a target cell and a caspase-related apoptosis-inducing molecule.

5. The chimeric protein of claim 4, wherein the protein selectively binds to an activated lymphocyte and the apoptosis inducing molecule is IL-2, IL-4, IL-8, IL-10, IL-12, IL-15, TNF-α, GM-CSF or TGF-β and the apoptosis inducing molecule is one of caspase 1 to 14, or a caspase-activating factor.

6. A method of inducing allograft tolerance comprising the administration of an effective amount of the chimeric protein of claim 5.

7. A method of purging a subject of activated lymphocytes comprising contacting the subject's lymphocytes with the chimeric protein of claim 5 ex vivo and returning the purged lymphocytes to the subject.

8. The method according to claim 7, wherein the subject has an autoimmune disease or allergies.

9. An isolated and purified nucleic acid encoding a polypeptide, wherein the polypeptide comprises a receptor-binding means and an apoptosis-inducing means.

10. The nucleic acid of claim 9, wherein the receptor-binding means comprises IL-2, Il-4, IL-8, IL-10, IL-12, IL-15, TNF-α, GM-CSF, or TGF-β.

11. The nucleic acid of claim 9, wherein the apoptosis-inducing means comprises any of caspases 1 to 14 or a caspase activating factor.

12. A polypeptide comprising a receptor-binding means and an apoptosis-inducing means.

13. The polypeptide of claim 12, wherein the receptor-binding means comprises IL-2, Il-4, IL-8, IL-10, IL-12, IL-15, TNF-α, GM-CSF, or TGF-β.

14. The polypeptide of claim 12, wherein the apoptosis-inducing means comprises any of caspases 1 to 14 or a caspase activating factor.

15. The polypeptide of claim 12, wherein the apoptosis-inducing means induces apoptosis in a cell after the polypeptide has been internalized into the cell.

16. A cell comprising the nucleic acid of claim 9.

17. A cell comprising the polypeptide of claim 12.

18. A matrix comprising the polypeptide of claim 12.

19. A method of inducing apoptosis in a cell comprising contacting the cell with the nucleic acid of claim 9.

20. A method of inducing apoptosis in a cell comprising contacting the cell with the polypeptide of claim 12.

21. A method of inducing apoptosis in a cell comprising contacting the cell with the cell of claim 16.

22. A method of inducing apoptosis in a cell comprising contacting the cell with the cell of claim 17.

23. A method of inducing apoptosis in a cell comprising contacting the cell with the matrix of claim 18.

24. The method of claim 19, 20, 21, 22 or 23, wherein the cell is a lymphocyte or a neoplastic cell.

25. The method of claim 24, wherein the lymphocyte is an activated T lymphocyte.

26. The method of claim 25, wherein the activated T lymphocyte is an activated T lymphocyte in, on, or from a human subject.

27. The method of claim 26, wherein the subject is in need of relief from any of the group consisting of autoimmune disease, graft-versus-host disease, allograft rejection, and xenograft rejection.

28. The method of claim 26, wherein the subject is at risk of autoimmune disease, graft-versus-host disease, allograft rejection, or xenograft rejection.

29. The method of claim 24, wherein the neoplastic cell is a human neoplastic cell.
